(19) United States
(12) Patent Application Publication
(10) Pub. No.: US 2009/0148413 A1
(43) Pub. Date: Jun. 11, 2009

(54) Method for treatment of irritable bowel syndrome

(76) Inventor: George Halow, El Paso, TX (US)

Correspondence Address: David G. Moore;Strasburger & Price, LLP, Suite 4400, 901 Main St., Dallas, TX, 75202, US

(21) Appl. No.: 12/001,603

(22) Filed: Dec. 11, 2007

(51) Int. Cl. A61K 45/00; A61P 1/00

(52) U.S. Cl. 424/93.4

(57) ABSTRACT

A composition and method for treatment for bowel disorders is described. The composition consists of three parts. The first part is a highly purified, insoluble, chemically unmodified fiber from plants which is mixed with an aqueous solution such as water. The second part of the composition is a commercially available probiotic preparation. These commercially probiotic preparations contain large numbers of viable microorganisms of either Lactobacillus sp., Bifidobacter sp., Streptococcus sp. or yeast. The third part of the composition is polyethylene glycol which may be administered simultaneously with the aforementioned fiber. Consumption of the fiber and aqueous solution combination plus probiotic and PEG by patients with constipation or Irritable Bowel Syndrome results in either a resolution of the symptoms or a marked improvement in symptoms without significant side effects. The disclosed combination and treatment method may be administered to patients with constipation and Irritable Bowel Syndrome for long periods of time without significant complications.

### CROSS-REFERENCE TO RELATED APPLICATIONS

None.

### STATEMENT REGARDING FEDERALLY SPONSORED RESEARCH OR DEVELOPMENT

The invention described in this application was not the subject of federally sponsored research or development.

### BACKGROUND

1. Field

The field of the invention disclosed in this application is a method for medical treatment of constipation and Irritable Bowel Syndrome. Specifically, a method employing a probiotic preparation in combination with fiber and polyethylene glycol (PEG) for the treatment of constipation and Irritable Bowel Syndrome is disclosed.

2. Description of the Related Art

The alimentary tract of most mammals consists of a long tube through the body. The alimentary tract begins at the mouth which is connected to the stomach by the esophagus. The stomach in turn is connected to the small intestine which is subsequently attached to the large intestine or colon. The colon ends at the rectum. Food enters the alimentary tract through the mouth and travels through the esophagus to the stomach where acids and various enzymes break down the food. Nutrients are absorbed in the small intestine and water is removed from the digested food by the colon. The colon also packages the digested food into mostly solid parcels for exit through the rectum.

In both the large and small intestine there exist enormous numbers of bacteria of many varieties. Because of the large diversity of organisms, no single organism is permitted to become dominant. In this way the intestines are protected from invasion by pathogenic microorganisms. The microorganisms which reside in the intestines also provide beneficial effects for the host organism. These beneficial effects include breaking down ingested food into component parts for ease of assimilation by the host. The bacteria also use the food ingested by the host to produce valuable nutrients such as vitamins which are absorbed by the body. When this delicate balance of diversity of microorganisms is disturbed, the effects may be transferred to the host in the form of sickness and disease.

Probiotic preparations are mixtures of viable microorganisms, mainly bacteria which commonly reside in the intestines, and which have been packaged in such a manner so as to be able to be easily ingested by a mammalian host. Probiotic preparations have been used to restore the diversity of the microbial ecology in the intestines by ingestion of preparations containing various types of viable bacteria. It is anticipated that this ingestion of the probiotic preparations will restore the diversity of microbes present in the intestines and promote intestinal health and well-being.

Probiotics have also been used to treat specific intestine-related diseases. In a patent to Neeser et al. (U.S. Pat. No. 6,835,376) a probiotic preparation of Lactobacillus paracasei was used to treat diarrhea by preventing the pathogenic bacteria from attaching to the cells lining the intestine, a prerequisite for the development of diarrhea caused by many bacteria.

In a patent issued to Perry (U.S. Pat. No. 6,203,797) a dietary supplement was prepared which, optionally, could contain probiotic preparations of “friendly” bacteria such as Lactobacillus bulgaricus, Lactobacillus acidophilus, Lactobacillus plantarum and Bifidobacterium bifidum.

A probiotic preparation of Enterococcus faecium, was used to treat Irritable Bowel Syndrome in a patent issued to Alleb et al. (U.S. Pat. No. 5,589,168)

Uremia related to kidney failure was treated with probiotic preparations consisting of ammoniaphilic bacteria, and sorbents to remove toxins in a patent issued to Ranganathan et al. (U.S. Pat. No. 6,706,287)

Constipation occurs when the stool is packed so tightly as to make it difficult or painful to excrete the feces. Constipation is usually treated with increased fluid consumption and fiber to soften the stool thus permitting the stool to travel through the colon to excretion.

Irritable Bowel Syndrome (IBS) is a chronic disease unrelated to common causes of diarrhea or constipation even though diarrhea and constipation may be a symptom of Irritable Bowel Syndrome. Symptoms of Irritable Bowel Syndrome include diarrhea, constipation, abdominal pain, gas and bloating of more than three months duration. Some patients may alternate bouts of constipation and diarrhea. It is unknown what causes Irritable Bowel Syndrome. There is some evidence to suggest that IBS is caused by infection. Other evidence suggests that IBS may be due to a lack of coordinated movement of the intestines. If the intestines move too quickly, food passes through the intestines more quickly and diarrhea results. If the muscles contract more slowly, the transit time of food through the intestines is increased resulting in constipation.

Diagnosis of Irritable Bowel Syndrome by medical doctors is typically accomplished by exclusion of other causes. Symptoms of IBS may last for many years in some patients. Treatments such as antibiotics, antipsychotics, diet, hypnotics and acupuncture have been tried as treatment for IBS with no consistent success. What is needed in the art is an effective and economical treatment for constipation and Irritable Bowel Syndrome.

### BRIEF SUMMARY OF THE INVENTION

The invention disclosed herein is an effective and economical treatment for constipation and Irritable Bowel Syndrome. The embodiments of the invention disclosed herein relate to the treatment of Irritable Bowel Syndrome and constipation using a mixture of highly purified fiber, various probiotic preparations and PEG. Conditions such as constipation following antibiotic therapy, or secondary to surgery are efficiently relieved by administration of a solution of highly purified fiber, probiotic preparations and PEG. Irritable Bowel Syndrome, also responds favorably following administration of probiotic with a solution of fiber and PEG. The fiber, the PEG and the probiotic preparations are commercially available thereby enabling the novel treatment disclosed herein to be administered quickly, easily and economically.

### BRIEF DESCRIPTION OF THE DRAWINGS

None.

### DETAILED DESCRIPTION OF THE INVENTION

Disorders of bowel function can be manifested by protean symptoms. Pain, bloating, gas, diarrhea, constipation and bleeding, among other symptoms, can all be manifestations of a bowel disorder. Some bowel disorders are iatrogenic. Constipation can result from prolonged antibiotic therapy or constipation may occur after surgery. Other bowel conditions can be chronic in duration. Irritable Bowel Syndrome constitutes a constellation of symptoms related to bowel dysfunction.

Many types of treatment have been tried in order to alleviate suffering caused by bowel disorders. Most of these treatments have been unsuccessful or only partially successful at alleviating the discomfort caused by these conditions. The invention disclosed herein is a composition and method for the treatment for constipation and Irritable Bowel Syndrome. The composition and method disclosed herein employs the administration of fiber in conjunction with probiotic preparations and PEG to individuals suffering from constipation and/or Irritable Bowel Syndrome.

The fiber used in the disclosed invention is highly-purified, chemically unmodified and suspended in an aqueous solution such as water or fruit juice to improve palatability. The fiber is plant-derived cellulose which is insoluble or only partially soluble in aqueous solutions. Approximately 10-25 grams of the aforementioned fiber are suspended in a large glass of water, for example, and consumed one time per day. The quantity of water needed in the preferred embodiment is, at a minimum, in the range of about 30 to about 60 milliliters.

In other embodiments of the invention disclosed herein, commercially-available sources of fiber may be used in the amounts described on the package label. Such other sources of fiber include for example:
1. Algin® (Greatest Herbs on Earth) is a nondigestible fiber derived from kelp or brown seaweed. Two capsules taken three times daily with two glasses of juice is the standard dosage. Each capsule contains 475 milligrams of sodium alginate.
2. Benefiber® (Novartis Consumer Health, Inc.) is a nonbulking fiber made from wheat dextrin. Benefiber® is gluten free and taken at the rate of 2 teaspoons of Benefiber suspended in 4-8 ounces of aqueous media three times a day.
3. Fibersure® (Procter & Gamble) is a fiber product composed of inulin. The standard dosage is 5 grams Fibersure® in one glass of water or other aqueous fluid.
4. Metamucil® (Proctor & Gamble) is a fiber product made from Psyllium husk and ingested at the rate of one teaspoon in 8 ounces of aqueous media, three times per day.

The probiotic preparation is commercially available and is taken according to manufacturer's instructions. Such commercially available probiotic preparations include for example:
1. CP-1 (Custom Probiotics, Inc. ) CP-1 is the trademark for a probiotic preparation including Lactobacillus acidophilus, Lactobacillus plantarum, Lactobacillus rhamnosus, Bifidobacterium bifidum and Bifidobacterium longum. Information provided by the manufacturer indicates that 2 capsules of CP-1 probiotic should be taken per day. Each capsule contains 35 billion microorganisms.
2. Culturelle® (Amerifit Nutrition, Inc.) Culturelle is the trademark for a probiotic preparation including composed of Lactobacillus GG with 10 billion viable bacteria per capsule. One-two capsules per day constitute the ordinary dosage of the Culturelle probiotic preparation.
3. Flora Q® (Kenwood Therapeutics) Flora Q is the trademark for a probiotic including Bifidobacterium, Lactobacillus acidophilus, Lactobaccillus paracasei and Streptococcus thermophilus. One capsule of the Flora Q probiotic product containing no less than 4 million CFU/ml (colony forming units/milliliter) is the standard daily dosage.
4. Florastor®E (Bicodex, Inc.) Florastor is the trademark for a probiotic preparation including lyophilized Saccharomyces boulardii, a yeast. One capsule, containing 5 billion live cells per capsule, taken twice daily is the standard dosage of the Florastor probiotic preparation.
5. HLC High Potency capsules (Emerson Ecologics, Inc.) HLC High Potency is the trademark for capsules of a probiotic preparation including two different strains of Lactobacillus acidophilus, and one strain each of Bifidobacterium bifidum and Bifidobacterium lactis. Each capsule of the HLC High Potency probiotic preparation contains 8 billion viable bacteria. Two capsules per day taken with meals is the normal dosage.
6. Lacidofil DF® (Xymogen, Inc.) Lacidofil DF is the trademark for a probiotic preparation including Bifidobacterium longum, Lactobacillus acidophilus and Lactobacillus rhamnosus. Each capsule of the Lacidofil DF probiotic preparation contains 2 billion live organisms. One to two capsules per day are taken before or during meals.
7. Ultra Flora plus DF® (Metagenics) Ultra Flora plus DF is the trademark for a probiotic preparation including Bifidobacterium lactis and Lactobacillus acidophilus. Each capsule of the Ultra Flora plus DF probiotic preparation contains no less than 5 billion live organisms. The normal dosage is 1-2 capsules per day.
8. VSL#3 (Sigma-Tau Pharmaceuticals, Inc.) VSL#3 is the trademark for a probiotic preparation including Bifidobacterium breve, Bifidobacterium infantis, Bifidobacterium longum, Lactobacillus acidophilus, Lactobacillus bulgaricus, Lactobacillus casei, Lactobacillus plantarum, and Streptococcus thermophilus. Each packet of the VSL#3 probiotic preparation contains 450 billion live lactic acid bacteria. One/half to 8 packets per day is the normal dosage.

Polyethylene glycol (PEG) is added to the fiber/probiotic treatment regimen described above. Polyethylene glycol (PEG) in an amount of about 10 grams to about 25 grams may be administered once per day as a standard dosage. It may be most convenient to mix the aforementioned amount of PEG with the 10-25 grams of fiber suspended in a minimum of about 30 to about 60 milliliters of aqueous media as described above. In this way the requisite amount of PEG and fiber could be simultaneously administered to the patient.

Approximately 10-25 grams of highly purified fiber which is plant derived, chemically unmodified and insoluble or partially insoluble in aqueous media such as water is suspended in a minimum of about 30 to about 60 milliliters of the aqueous media. Fruit juice may be substituted for water to improve palatability of the fiber. This quantity of fiber is consumed one time per day. In addition, one of the commercially available probiotic preparations is also ingested. The daily dosage of the probiotic preparation to be administered with the fiber is either 2 capsules of CP-1, 1-2 capsules of Culturelle®, Flora Q® (1 capsule), Florastor® (1 capsule, twice per day), HLC (2 capsules with meals), Lacidofil® (1-2 capsules prior to or with meals), Ultraflora® (1-2 capsules) or VSC#3 (0.5-8 packets). Alternatively, a commercially-available form of fiber ingested in the amounts described by the manufacturer may be substituted for the aforementioned fiber preparation. Polyethylene glycol is added to the fiber/probiotic preparation composition in the amounts described above. The above described therapeutic combination incorporating fiber, a probiotic preparation and PEG are used in each of the embodiments of the disclosed invention as described below.

### First Embodiment

Patients undergoing surgery frequently have narcotics and other medicines administered prior to, and during surgery to relieve pain and relax muscles. A common side effect of these medicines, especially the narcotic medicines, is to decrease motility of the intestine resulting in constipation. This constipation following surgery is usually self-limiting. However, in some circumstances the constipation can be severe, long-lasting and result in fecal impactions and intestinal obstruction requiring medical/surgical intervention. Fiber has been used as a treatment for constipation. However, according to the disclosed invention, fiber in conjunction with commercially-available probiotic preparations and PEG is administered to patients with post-surgical constipation. Patients with post-surgical constipation who receive the aforementioned administration of fiber, a probiotic preparation and PEG are relieved of the constipation quicker than with just fiber alone. Further, these post surgical patients who are administered fiber plus a probiotic preparation and PEG recover from their constipation with few, if any, side effects.

### Second Embodiment

Patients undergoing long-term or chronic antibiotic therapy frequently develop constipation presumably because of the disruption in the diversity of the microbial flora of the intestine caused by the administration of the antibiotic. Fiber has been used alone to treat the symptoms of constipation in patients with antibiotic associated constipation. However, fiber plus a probiotic preparation and PEG not only relieves the symptoms of constipation but also helps quickly restore the normal balance of microbial diversity in the intestines. Patients receiving fiber, a probiotic preparation and PEG have fewer relapses of constipation than those individuals receiving fiber alone.

### Third Embodiment

Patients with Irritable Bowel Syndrome manifest a constellation of symptoms including constipation, diarrhea, gas, bloating abdominal pain and bleeding. These symptoms may last for months or years. Attempts to treat Irritable Bowel Syndrome have included antibiotics, antidepressants, acupuncture and others with inconsistent success. Treatment of patients suffering from Irritable Bowel Syndrome with fiber plus a probiotic preparation and PEG as disclosed herein results in resolution of the symptoms of IBS without any significant complications. The fiber plus a probiotic preparation and PEG disclosed herein may be administered for long periods of time without causing significant complications.

There are other bowel disorders and other combinations of fiber plus a probiotic preparation and PEG not specifically disclosed herein. A person of ordinary skill in the art would recognize that such combinations exist. Those combinations even though not specifically disclosed herein are included within the scope and meaning of the claims appended hereto.

I claim:

1. A treatment for constipation and Irritable Bowel Syndrome in mammals using a composition comprising:
an aqueous media containing fiber;
a probiotic preparation; and
polyethylene glycol.

2. The treatment for constipation and Irritable Bowel Syndrome as defined in claim 1 wherein said fiber is chemically unmodified.

3. The treatment for constipation and Irritable Bowel Syndrome as defined in claim 1 wherein said fiber is insoluble or partially insoluble in said aqueous media.

4. The treatment for constipation and Irritable Bowel Syndrome as defined in claim 1 wherein said fiber is plant derived cellulose.

5. The treatment for constipation and Irritable Bowel Syndrome as defined in claim 1 wherein approximately 10-25 grams of said fiber is suspended in at least about 30 to about 60 milliliters of said aqueous media.

6. The treatment for constipation and Irritable Bowel Syndrome as defined in claim 1 wherein said probiotic preparation is designated by the trademark CP-1 and is taken in a dosage of about two capsules per day.

7. The treatment for constipation and Irritable Bowel Syndrome as defined in claim 1 wherein said probiotic preparation is designated by the trademark Culturelle® and is taken in a dosage of about 1-2 capsules per day.

8. The treatment for constipation and Irritable Bowel Syndrome as defined in claim 1 wherein said probiotic preparation is designated by the trademark Flora Q® and is taken in a dosage of about one capsule per day.

9. The treatment for constipation and Irritable Bowel Syndrome as defined in claim 1 wherein said probiotic preparation is designated by the trademark Florastor® and is taken in a dosage of about two capsules per day.

10. The treatment for constipation and Irritable Bowel Syndrome as defined in claim 1 wherein said probiotic preparation is designated by the trademark High Potency HLC and is taken in a dosage of two capsules per day.

11. The treatment for constipation and Irritable Bowel Syndrome as defined in claim 1 wherein said probiotic preparation is designated by the trademark Lacidofil® and is taken in a dosage of about one to two capsules per day.

12. The treatment for constipation and Irritable Bowel Syndrome as defined in claim 1 wherein said probiotic preparation is designated by the trademark Ultraflora plus DF® and is taken in a dosage of about 1-2 capsules per day.

13. The treatment for constipation and Irritable Bowel Syndrome as defined in claim 1 wherein said probiotic preparation is designated by the trademark VS #3 and is taken in a dosage of about one half to eight packets per day.

14. The treatment for constipation and Irritable Bowel Syndrome as defined in claim 1 wherein said polyethylene glycol is administered in a daily dosage of about 10 grams to about 25 grams suspended in an aqueous medium.

15. The treatment for constipation and Irritable Bowel Syndrome as defined in claim 1 wherein the treatment is given to a human being.

16. A method for treating mammals with constipation and Irritable Bowel Syndrome comprising the steps of:
mixing fiber and PEG in an aqueous media; and
administering a probiotic preparation and said fiber/PEG solution to those mammals exhibiting symptoms of constipation and Irritable Bowel Syndrome.

17. The method for treating mammals with constipation and Irritable Bowel Syndrome as defined in claim 16 wherein said fiber is unmodified.

18. The method for treating mammals with constipation and Irritable Bowel Syndrome as defined in claim 16 wherein said fiber is either insoluble or partially soluble in aqueous media.

19. The method for treating a mammal with constipation and Irritable Bowel Syndrome as defined in claim 16 wherein said fiber is plant derived cellulose.

20. The method for treating a mammal with constipation and Irritable Bowel Syndrome as defined in claim 16 wherein about 10 grams to about 25 grams of said fiber is suspended in at least about 30 to about 60 milliliters of aqueous media.

21. The method for treating a mammal with constipation and Irritable Bowel Syndrome as defined in claim 16 wherein said probiotic preparation is designated by the trademark CP-1 and is taken in a dosage of about two capsules per day.

22. The method for treating a mammal with constipation and Irritable Bowel Syndrome as defined in claim 16 wherein said probiotic preparation is designated by the trademark Culturelle® and is taken in a dosage of about 1-2 capsules per day.

23. The method for treating a mammal with constipation and Irritable Bowel Syndrome as defined in claim 16 wherein said probiotic preparation is designated by the trademark Flora Q® and is taken in a dosage of about one capsule per day.

24. The method for treating a mammal with constipation and Irritable Bowel Syndrome as defined in claim 16 wherein said probiotic preparation is designated by the trademark Florastor® and is taken in a dosage of about two capsules per day.

25. The method for treating a mammal with constipation and Irritable Bowel Syndrome as defined in claim 16 wherein said probiotic preparation is designated by the trademark HLC high potency and is taken in a dosage of two capsules per day.

26. The method for treating a mammal with constipation and Irritable Bowel Syndrome defined in claim 16 wherein said probiotic preparation is designated by the trademark Lacidofil® and is taken in a dosage of about one to two capsules per day.

27. The method for treating a mammal with constipation and Irritable Bowel Syndrome as defined in claim 16 wherein said probiotic preparation is designated by the trademark UltraFlora plus DF® and is taken in a dosage of about 1-2 capsules per day.

28. The method for treating a mammal with constipation and Irritable Bowel Syndrome as defined in claim 16 wherein said probiotic preparation is designated by the trademark VSC #3 and is taken in a dosage of about one half to eight packets per day.

29. The method for treating a mammal with constipation and Irritable Bowel Syndrome as defined in claim 16 wherein about 10 grams to about 25 grams of PEG suspended in an aqueous media is administered to the mammals.

30. The method for treating a mammal with constipation and Irritable Bowel Syndrome as defined in claim 16 wherein said method of treatment is given to a human being.
